(19) United States
(12) Patent Application Publication
(10) Pub. No.: US 2015/0045865 A1
(43) Pub. Date: Feb. 12, 2015

(54) SYSTEMS AND METHODS FOR MAKING AND USING LEAD ANCHORS FOR LEADS OF ELECTRICAL STIMULATION SYSTEMS

(71) Applicant: BOSTON SCIENTIFIC NEUROMODULATION CORPORATION, Valencia, CA (US)

(72) Inventors: Ranjan Krishna Mukhari Nageri, Valencia, CA (US); Geoffrey Abellana Villarta, Valencia, CA (US)

(21) Appl. No.: 14/452,467

(22) Filed: Aug. 5, 2014

Related U.S. Application Data
(60) Provisional application No. 61/863,137, filed on Aug. 7, 2013.

(51) Int. Cl. A61N 1/05

(52) U.S. Cl. CPC A61N 1/05
USPC 607/116

(57) ABSTRACT

A lead anchor includes a lead lumen forming a continuous passageway through an inner housing. A fastener lumen extends along the inner housing and forms an intersection with the lead lumen. A lead-retention assembly removably retains a lead within the lead anchor. The lead-retention assembly includes a sleeve formed from a rigid material. The sleeve is disposed within the lead lumen at the intersection between the fastener lumen and the lead lumen. The sleeve lumen receives the lead when the lead is received by the lead lumen. A fastener is disposed in the fastener lumen and retains the received lead within the lead anchor by pressing against the sleeve to reduce the diameter of the sleeve at the intersection between the fastener lumen and the lead lumen by reducing the width of a longitudinal cutout defined along the sleeve at the intersection between the fastener lumen and the lead lumen.

### CROSS-REFERENCE TO RELATED APPLICATIONS

This application claims the benefit under 35 U.S.C. §119(e) of U.S. Provisional Patent Application Ser. No. 61/863,137, filed Aug. 7, 2013, which is incorporated herein by reference.

### FIELD

The present invention is directed to the area of implantable electrical stimulation systems and methods of making and using the systems. The present invention is also directed to implantable electrical stimulation systems that include lead anchors for anchoring leads to patient tissue, as well as methods of making and using the leads, lead anchors, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a lead anchor includes an inner housing having an outer surface, a top end, a first end, and a second end opposite to the first end. The inner housing defines a lead lumen forming a continuous passageway through the inner housing. The lead lumen has a first opening defined along the first end of the inner housing and a second opening defined along the second end of the inner housing. A fastener lumen extends from the top end of the inner housing and forms an intersection with the lead lumen. An exterior covering is disposed over the outer surface of the inner housing. A lead-retention assembly is configured and arranged for removably retaining an electrical stimulation lead within the lead anchor. The lead-retention assembly includes a sleeve formed from a rigid material and having a first end portion, an opposing second end portion, a diameter, and a longitudinal length. The sleeve is disposed along surfaces of the lead lumen at the intersection between the fastener lumen and the lead lumen. The sleeve defines a sleeve lumen and at least one longitudinal cutout having a cutout width and extending along a portion of the longitudinal length of the sleeve. The sleeve lumen is configured and arranged to receive the electrical stimulation lead when the electrical stimulation lead is received by the lead lumen. A fastener is disposed in the fastener lumen and is configured and arranged for retaining the received electrical stimulation lead within the lead anchor by pressing against the sleeve to reduce the diameter of the sleeve at the intersection between the fastener lumen and the lead lumen by reducing the width of the longitudinal cutout at the intersection between the fastener lumen and the lead lumen.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system that includes a paddle lead electrically coupled to a control module, according to the invention;

FIG. 2 is a schematic view of one embodiment of an electrical stimulation system that includes a percutaneous lead electrically coupled to a control module, according to the invention;

FIG. 3A is a schematic view of one embodiment of the control module of FIG. 1 configured and arranged to electrically couple to an elongated device, according to the invention;

FIG. 3B is a schematic view of one embodiment of a lead extension configured and arranged to electrically couple the elongated device of FIG. 2 to the control module of FIG. 1, according to the invention;

FIG. 4A is a schematic perspective view of one embodiment of an inner housing, a sleeve, and a fastener suitable for use in a lead anchor, the sleeve disposed in the inner housing and suitable for receiving a portion of a lead body, and the fastener suitable for securing the received lead body to the inner housing by tightening against the sleeve, according to the invention;

FIG. 4B is a schematic perspective, longitudinal cross-sectional view of one embodiment of the inner housing, sleeve, and fastener of FIG. 4A, according to the invention;

FIG. 4C is a schematic longitudinal cross-sectional view of one embodiment of the inner housing, sleeve, and fastener of FIG. 4A, according to the invention;

FIG. 5A is a schematic perspective, longitudinal cross-sectional view of another embodiment of a sleeve disposed in the inner housing of FIGS. 4A-4C, the sleeve suitable for receiving a portion of a lead body, the sleeve extending outwardly from the inner housing on opposing ends, the sleeve also defining strain-relief grooves extending along a portion of the sleeve extending from the inner housing, according to the invention;

FIG. 5B is a schematic perspective view of yet another embodiment of a sleeve disposed in the inner housing of FIGS. 4A-4C, the sleeve suitable for receiving a portion of a lead body, the sleeve extending outwardly from the inner housing on opposing ends, the sleeve also defining strain-relief grooves extending along a portion of the sleeve extending from the inner housing, the inner housing shown as being transparent, according to the invention;

FIG. 6 is schematic perspective view of one embodiment of the inner housing and sleeve of FIG. 5B disposed within an exterior covering to form a lead anchor, the inner housing and exterior covering each shown as being transparent, according to the invention;

FIG. 7A is a schematic perspective view of another embodiment of a lead anchor suitable for receiving a lead body and coupling the received lead body to patient tissue, the lead anchor including an exterior covering with an elongated, tapered, first end portion and eyelets for facilitating securement of the lead anchor to patient tissue, according to the invention;

FIG. 7B is a schematic perspective view of one embodiment of the lead anchor of FIG. 7A with a transparent exterior covering, the lead anchor including a sleeve disposed in the inner housing of FIGS. 4A-4C, the sleeve defining strain-relief grooves extending along opposing first and second end portions of the sleeve extending outwardly from the inner housing, according to the invention;

FIG. 8A is a schematic perspective view of another embodiment of an inner housing and a fastener suitable for use in a lead anchor, the inner housing defining multiple lead lumens each suitable for receiving a portion of a different lead body, and the fastener suitable for securing each of the received lead bodies to the inner housing by tightening against the received portions of the lead bodies, the inner housing shown as being transparent, according to the invention;

FIG. 8B is a schematic end view of one embodiment of the inner housing and the fastener of FIG. 8A, the inner housing shown as being transparent, according to the invention;

FIG. 8C is a side, perspective longitudinal cross-sectional view of one embodiment of a lead anchor formed using the inner housing and the fastener of FIG. 8A, according to the invention;

FIG. 8D is a top, perspective longitudinal cross-sectional view of one embodiment of the lead anchor formed using the inner housing and the fastener of FIG. 8A, according to the invention; and

FIG. 9A is a schematic transverse cross-sectional view of another embodiment of the lead anchor of FIGS. 8C-8D that includes the inner housing, the fastener, and the exterior covering of FIGS. 8A-8B with sleeves disposed in lead lumens defined in the inner housing, the fastener in a disengaged position such that the fastener is untightened against the sleeves, according to the invention;

FIG. 9B is a schematic transverse cross-sectional view of one embodiment of the fastener, inner housing, and sleeves of FIG. 9A with the fastener in an engaged position such that the fastener is tightened against the sleeves, according to the invention; and

FIG. 10 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to implantable electrical stimulation systems that include lead anchors for anchoring leads to patient tissue, as well as methods of making and using the leads, lead anchors, and electrical stimulation systems.

Suitable implantable electrical stimulation systems include, but are not limited to, at least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; and 8,364,278; and U.S. Patent Application Publication No. 2007/0150036, all of which are incorporated by reference.

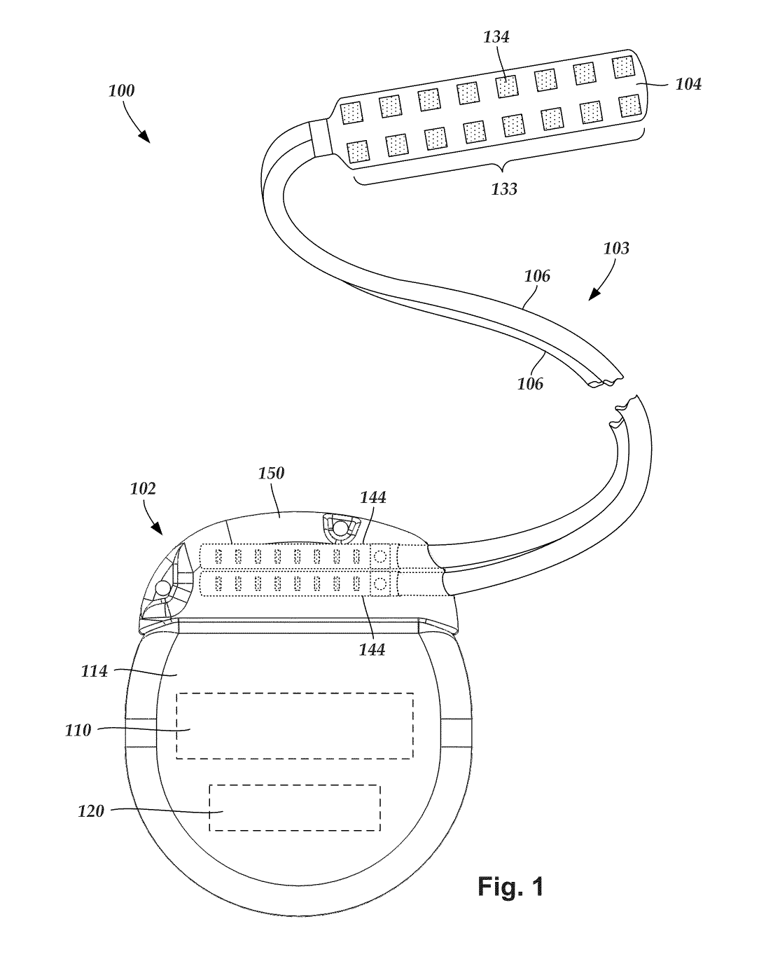

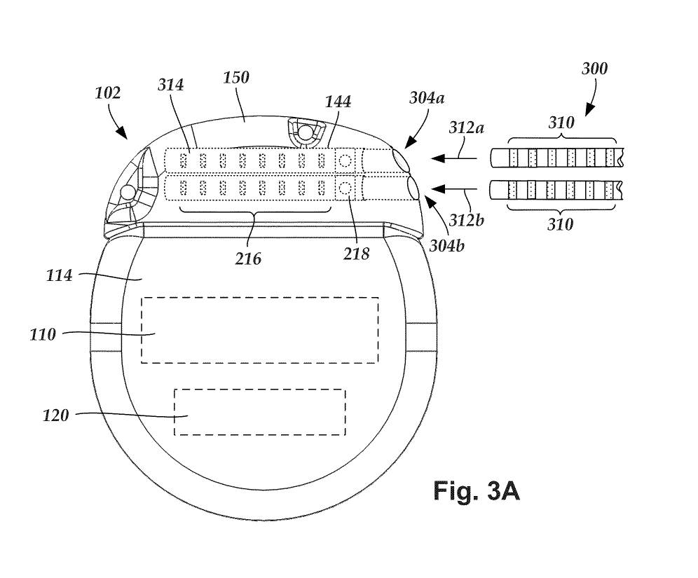

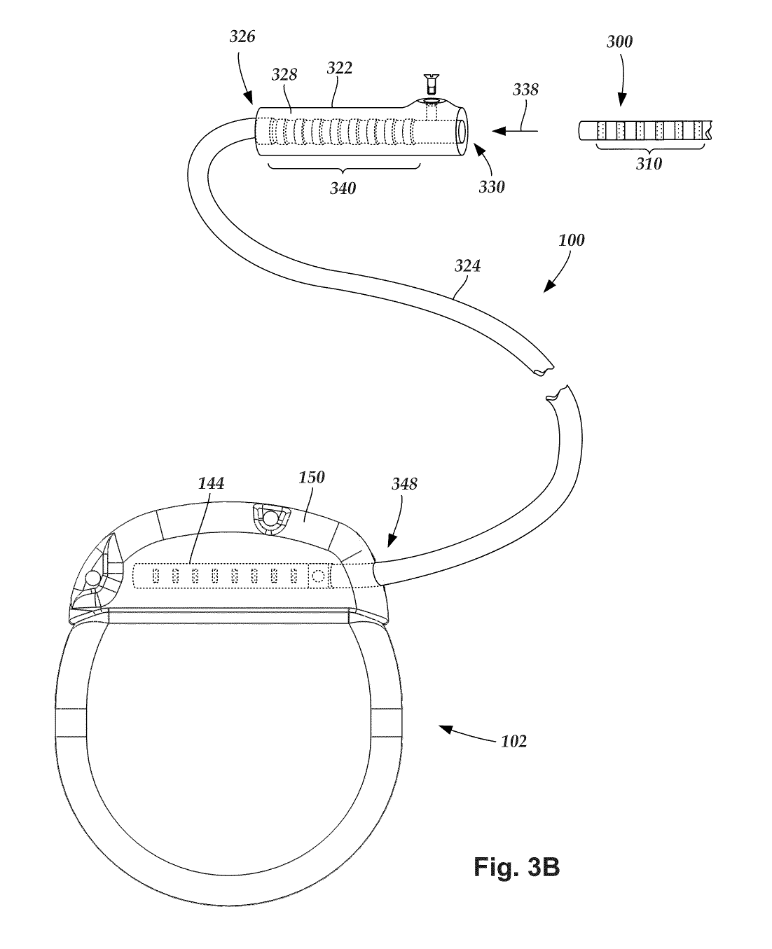

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 1, the lead 103 is shown having two lead bodies 106. It will be understood that the lead 103 can include any suitable number of lead bodies including, for example, one, two, three, four, five, six, seven, eight or more lead bodies 106. An array of electrodes 133, such as electrode 134, is disposed on the paddle body 104, and an array of terminals (e.g., 310 in FIG. 3A-3B) is disposed along each of the one or more lead bodies 106.

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

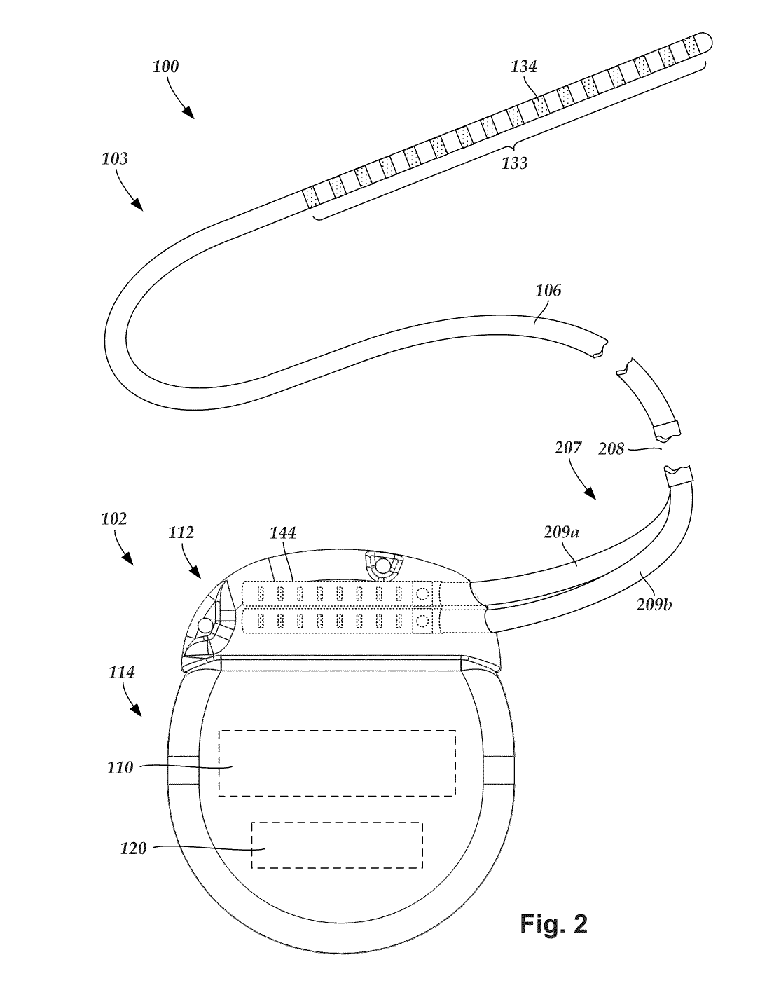

FIG. 2 illustrates schematically another embodiment of the electrical stimulation system 100, where the lead 103 is a percutaneous lead. In FIG. 2, the electrodes 134 are shown disposed along the one or more lead bodies 106. In at least some embodiments, the lead 103 is isodiametric along a longitudinal length of the lead body 106.

The lead 103 can be coupled to the control module 102 in any suitable manner. In FIG. 1, the lead 103 is shown coupling directly to the control module 102. In at least some embodiments, the lead 103 includes a single proximal end portion. In at least some other embodiments, the lead 103 includes two or more proximal end portions (“tails”).

In at least some other embodiments, the lead 103 couples to the control module 102 via one or more intermediate devices (300 in FIGS. 3A-3B). For example, in at least some embodiments one or more lead extensions 324 (see e.g., FIG. 3B) are disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 2, the electrical stimulation system 100 is shown having a splitter 207 configured and arranged for facilitating coupling of the lead 103 to the control module 102. The splitter 207 includes a splitter connector 208 configured to couple to a proximal end of the lead 103, and one or more splitter tails 209a and 209b configured and arranged to couple to the control module 102 (or another splitter, a lead extension, an adaptor, or the like).

The control module 102 typically includes a connector inner housing 112 and a sealed electronics inner housing 114. An electronic subassembly 110 and an optional power source 120 are disposed in the electronics inner housing 114. A control module connector 144 is disposed in the connector inner housing 112. The control module connector 144 is configured and arranged to make an electrical connection between the lead 103 and the electronic subassembly 110 of the control module 102.

The electrical stimulation system or components of the electrical stimulation system, including the paddle body 104, the one or more of the lead bodies 106, and the control module 102, are typically implanted into the body of a patient. The electrical stimulation system can be used for a variety of applications including, but not limited to deep brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium.

Any suitable number of electrodes 134 can be disposed on the lead including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. In FIG. 1, the electrodes 134 are arranged into two columns, where each column has eight electrodes 134.

The electrodes of the paddle body 104 (or one or more lead bodies 106) are typically disposed in, or separated by, a non-conductive, biocompatible material such as, for example, silicone, polyurethane, polyetheretherketone (“PEEK”), epoxy, and the like or combinations thereof. The one or more lead bodies 106 and, if applicable, the paddle body 104 may be formed in the desired shape by any process including, for example, molding (including injection molding), casting, and the like. The non-conductive material typically extends from the distal ends of the one or more lead bodies 106 to the proximal end of each of the one or more lead bodies 106.

In the case of paddle leads, the non-conductive material typically extends from the paddle body 104 to the proximal end of each of the one or more lead bodies 106. Additionally, the non-conductive, biocompatible material of the paddle body 104 and the one or more lead bodies 106 may be the same or different. Moreover, the paddle body 104 and the one or more lead bodies 106 may be a unitary structure or can be formed as two separate structures that are permanently or detachably coupled together.

Terminals (e.g., 310 in FIGS. 3A-3B) are typically disposed along the proximal end of the one or more lead bodies 106 of the electrical stimulation system 100 (as well as any splitters, lead extensions, adaptors, or the like) for electrical connection to corresponding connector contacts (e.g., 314 in FIGS. 3A-3B). The connector contacts are disposed in connectors (e.g., 144 in FIGS. 1-3B; and 322 FIG. 3B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 3A is a schematic side view of one embodiment of a proximal end of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 3B, an adaptor, or the like or combinations thereof), or a combination thereof.

The control module connector 144 defines at least one port into which a proximal end of the elongated device 300 can be inserted, as shown by directional arrows 312a and 312b. In FIG. 3A (and in other figures), the connector inner housing 112 is shown having two ports 304a and 304b. The connector inner housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end(s) of the elongated device(s) 300 to electrically couple the control module 102 to the electrodes (134 of FIG. 1) disposed on the paddle body 104 of the lead 103. Examples of connectors in control modules are found in, for example, U.S. Pat. Nos. 7,244,150 and 8,224,450, which are incorporated by reference.

FIG. 3B is a schematic side view of another embodiment of the electrical stimulation system 100. The electrical stimulation system 100 includes a lead extension 324 that is configured and arranged to couple one or more elongated devices 300 (e.g., one of the lead bodies 106 of FIGS. 1 and 2, the splitter 207 of FIG. 2, an adaptor, another lead extension, or the like or combinations thereof) to the control module 102. In FIG. 3B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144, or to receive multiple elongated devices 300, or both.

A lead extension connector 322 is disposed on the lead extension 324. In FIG. 3B, the lead extension connector 322 is shown disposed at a distal end 326 of the lead extension 324. The lead extension connector 322 includes a connector inner housing 328. The connector inner housing 328 defines at least one port 330 into which terminals 310 of the elongated device 300 can be inserted, as shown by directional arrow 338. The connector inner housing 328 also includes a plurality of connector contacts, such as connector contact 340. When the elongated device 300 is inserted into the port 330, the connector contacts 240 disposed in the connector inner housing 328 can be aligned with the terminals 310 of the elongated device 300 to electrically couple the lead extension 324 to the electrodes (134 of FIGS. 1 and 2) disposed along the lead (103 in FIGS. 1 and 2).

In at least some embodiments, the proximal end of the lead extension 324 is similarly configured and arranged as a proximal end of the lead 103 (or other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 are electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device). In other embodiments (and as shown in FIG. 3B), the proximal end 348 of the lead extension 324 is configured and arranged for insertion into the control module connector 144.

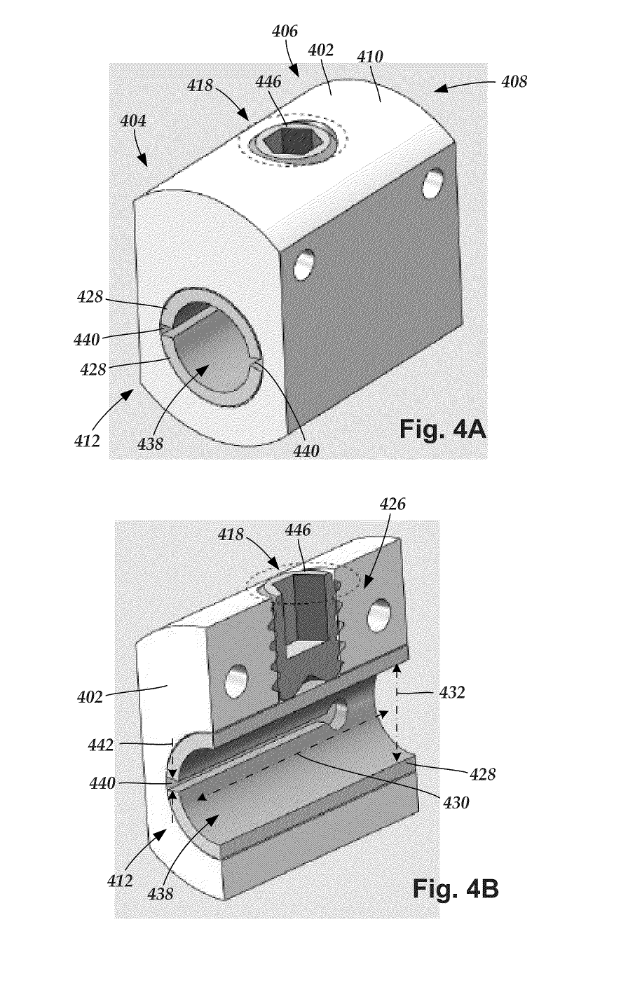

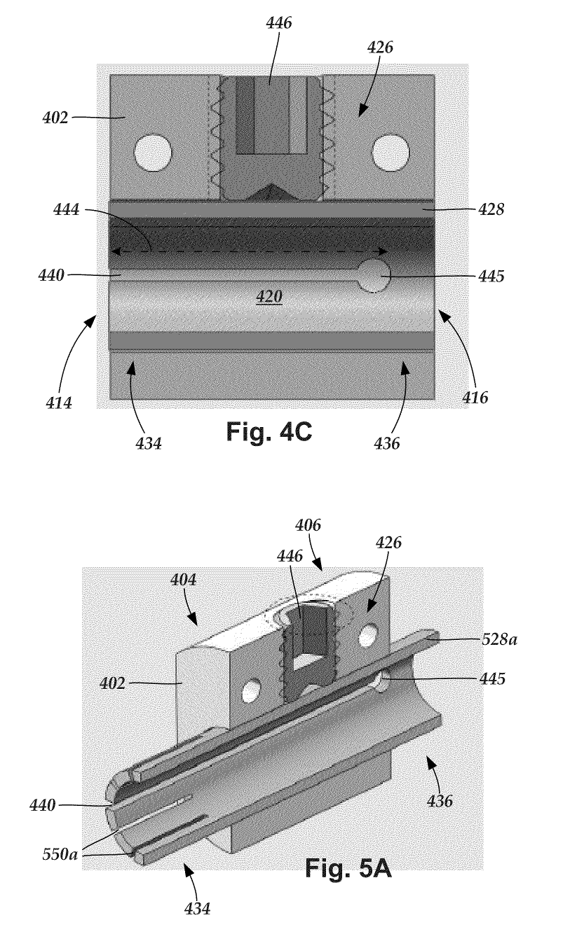

Turning to FIG. 4A, a lead anchor can be used in an implantable device, such as an implantable spinal cord stimulator, to anchor a lead to patient tissue. The lead anchor includes a fastener, which may be tightened to retain a portion of the lead body within the lead anchor.

Magnetic resonance imaging (“MRI”) is commonplace in many medical settings. Conventional implanted electrical stimulation systems are often incompatible MRI due to large radio frequency (“RF”) pulses used by MRI devices. The RF pulses can generate transient signals in the conductors and electrodes of an implanted lead.

These signals can have deleterious effects including, for example, unwanted heating of the tissue causing tissue damage, induced currents in the lead, or premature failure of electronic components. It may be useful to form a lead anchor such that it is compatible with MRI devices, as well as other devices that potentially expose a patient to RF irradiation.

A common cause of the electrical interaction between the electrical stimulation system and RF irradiation is common-mode coupling of the applied electromagnetic field. The interaction can be modeled as a series of distributed sources along the elongated conductive structures of the electrical stimulation system, such as leads, lead extensions, or conductors within leads or lead extensions. Common-mode induced RF currents may reach amplitudes of greater than one ampere in MRI environments. Such currents can cause heating and potentially disruptive voltages within electronic circuits.

To reduce the susceptibility of the electrical stimulation system to undesired RF irradiation, it may be advantageous to construct the lead anchor from one or more materials that reduce susceptibility of the lead anchor to undesired RF irradiation, while still maintaining appropriate biocompatibility for prolonged implantation and sufficient mechanical integrity to anchor a lead. Some exemplary materials include one or more polymers (e.g., polyetheretherketone, polyethersulfone, polyethylene, polypropylene, polyurethane, polyetherimide, polycarbonate, nylon, polysulfone, polymethylmethacrylate, or the like or combinations thereof), one or more ceramics, one or more non-magnetically reactive metals, or the like or combinations thereof.

The lead anchor includes an inner housing disposed in an exterior covering. The inner housing defines at least one lead lumen that extends across an entire length of the inner housing and at least one fastener lumen that intersects with the lead lumen. The lead lumen is suitable for receiving a portion of a lead body, and the fastener lumen is suitable for receiving a fastener that can tighten, either directly or indirectly, against the received portion of the lead body at the intersection between the fastener lumen and the lead lumen to retain the received portion of the lead body.

FIGS. 4A-4C illustrate one embodiment of an inner body suitable for use in a lead anchor. FIG. 4A illustrates, in perspective view, one embodiment of an inner housing 402. FIG. 4B illustrates, in perspective, longitudinal cross-section, one embodiment of the inner housing 402. FIG. 4C illustrates, in longitudinal cross-section, one embodiment of the inner housing 402.

The inner housing 402 has a first end 404, a second end 406 opposite to the first end 404, a top end 408, and an outer surface 410. The inner housing 402 includes a lead lumen 412, which provides a continuous passageway through the inner housing 402 between the first end 404 and the second end 406. The lead lumen 412 includes a first opening 414 defined along the first end 404 and a second opening 416 defined along the second end 406. The lead lumen 412 is dimensioned to receive a portion of a lead, such as the lead 103, from either of the first opening 414 or the second opening 416. In at least some embodiments, the lead lumen 412 receives the lead such that the lead extends from both the first opening 414 and the second opening 416.

The inner housing 402 defines a fastener lumen 418 that extends from the top end 408 into the inner housing 402 and that forms an intersection 420 with the lead lumen 412. The fastener lumen 418 may open along any suitable location on the outer surface 410 of the inner housing 402. In at least some embodiments, the fastener lumen 418 is transverse, or substantially transverse, to the lead lumen 412. The fastener lumen 418 may, optionally, be threaded, to receive a fastener that screws into the fastener lumen 418.

A lead-retention assembly 426 is configured and arranged for removably retaining a lead, such as the lead 103, within the lead anchor. The lead-retention assembly 426 includes a sleeve 428 and a fastener 446. The sleeve 428 is disposed along walls of the lead lumen 412 at the intersection 420 between the fastener lumen 418 and the lead lumen 412. An outer diameter of the sleeve 428 is sized to fit within the lead lumen 412, and an inner diameter 432 of the sleeve 428 is adapted to receive the lead.

The sleeve 428 has a longitudinal length 430, a first end portion 434, an opposing second end portion 436, and a sleeve lumen 438 extending along the entire longitudinal length 430 of the sleeve 428. The sleeve lumen 438 is dimensioned to receive an electrical stimulation lead, such as the lead 103, when the lead is received by the lead lumen 412. In at least some embodiments, the sleeve 428 is rigid such that the sleeve lumen 438 does not collapse when the lead is not received by the sleeve 428.

The sleeve 428 defines at least one longitudinal cutout 440 extending along a portion of the longitudinal length 430 of the sleeve 428 from the lateral edge of the first end portion 436 to a terminus 445. The longitudinal cutout 440 has a width 442 and a longitudinal length 444. In at least some embodiments, the sleeve 428 is rigid such that the width 442 of the longitudinal cutout 440 remains constant absent an applied force pressing against the sleeve 428.

In at least some embodiments, the longitudinal cutout 440 extends along at least 50%, 60%, 70%, 80%, 90%, or more of the longitudinal length 430 of the sleeve 428. In some embodiments, a single cutout 440 is defined along the sleeve 428. In alternate embodiments, multiple cutouts 440 are defined along the sleeve 428. In FIG. 4A, two cutouts 440 are shown defined along the sleeve 428, each extending parallel to each other and circumferentially offset from each other by 180°.

In at least some embodiments, the terminus 445 of the longitudinal cutout 440 has a width greater than the width 442 along the remaining portions of the longitudinal cutout 440. In at least some embodiments, the terminus 445 is a circular opening defined along the surface of the sleeve 428. In at least some other embodiments, the terminus 445 extends substantially along a circumference of the sleeve 428.

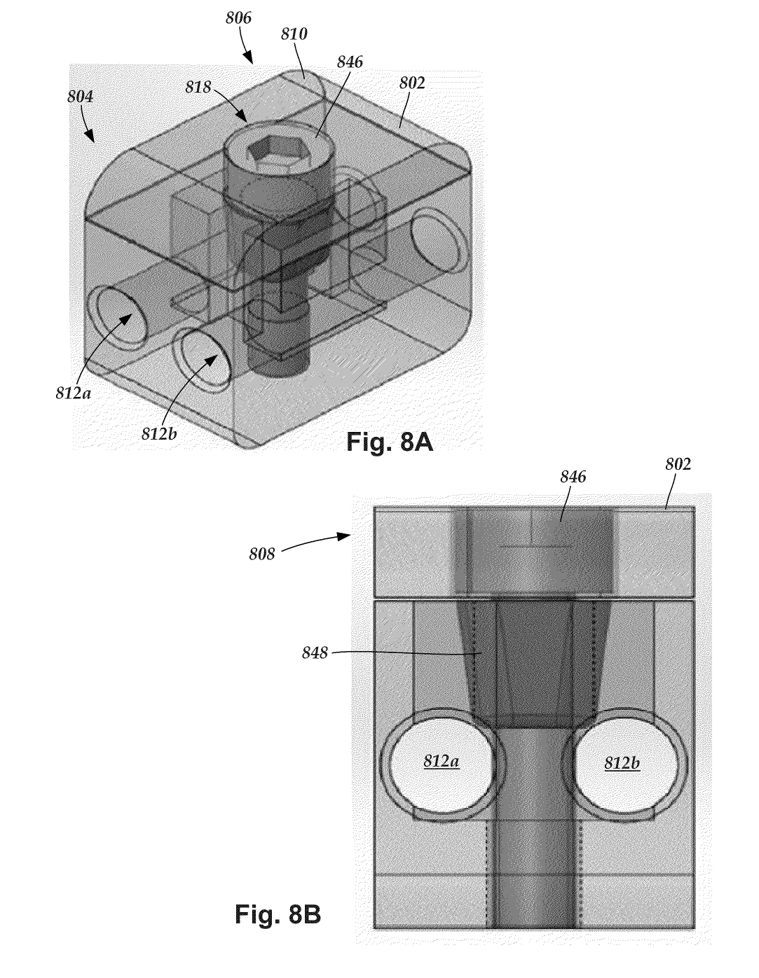

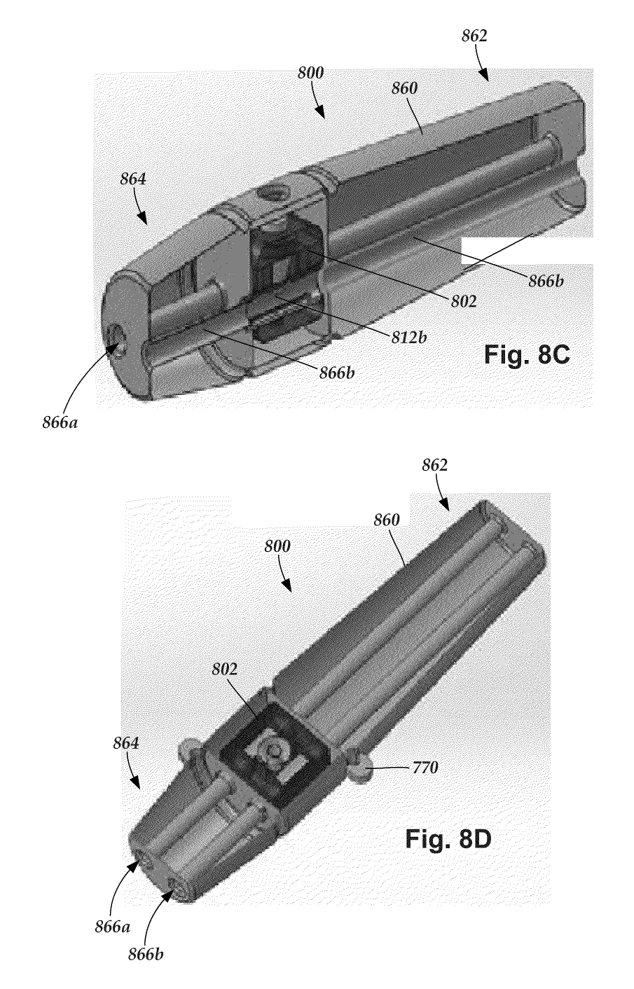

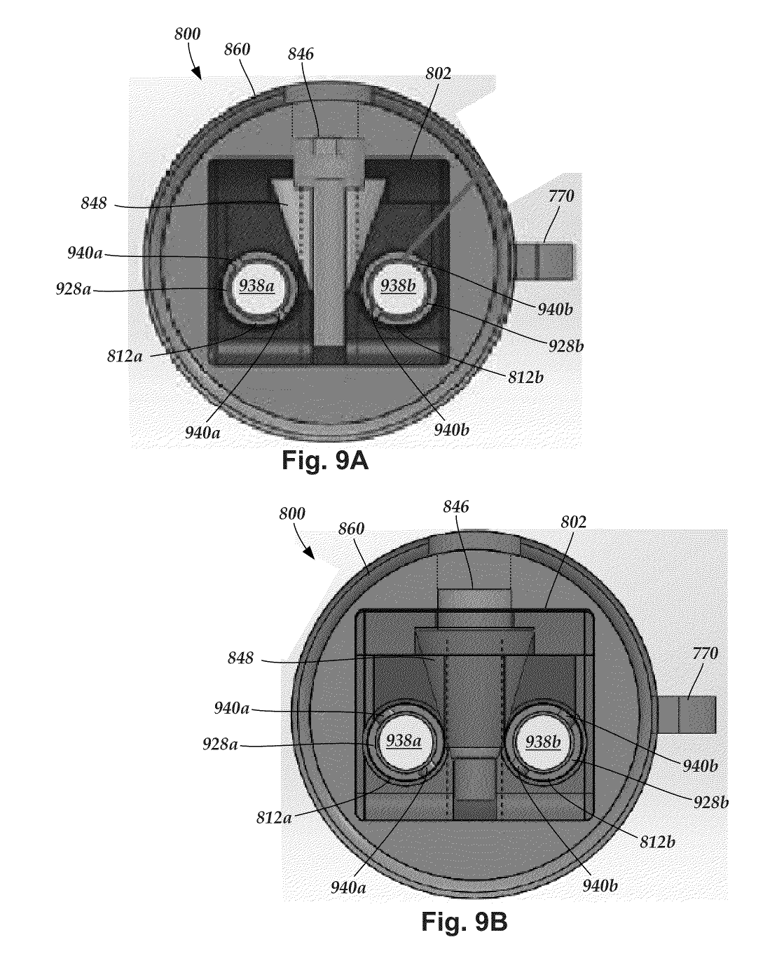

The fastener 446 of the lead-retention assembly 426 is insertable into the fastener lumen 418 and adapted for securing the sleeve 428 against the lead. The fastener 446 may be of a variety of shapes and sizes for insertion into the fastener lumen 418 and engagement with the sleeve 428. In some embodiments, the fastener 446 is a set screw suitable for extending along the fastener lumen 418 and engaging the sleeve 428. In at least some embodiments, the fastener 446 is formed as a plug with a tapered section (see e.g., FIGS. 8A-9B). In at least some embodiments, the fastener 446 is formed from one or more rigid, durable, biocompatible, MRI-safe materials.

The fastener 446 retains the received lead within the lead anchor by pressing against the sleeve 428 when inserted into the fastener lumen 418. Pressing the fastener 446 against the sleeve 428 causes the width 442 of the longitudinal cutout 440 to be reduced. The reduction of the width 442 of the longitudinal cutout 440 causes a corresponding reduction of the inner diameter 432 of the sleeve 428, thereby pressing the sleeve 428 against the received lead and securing the lead within the sleeve 428.

The sleeve 428 can have any suitable longitudinal length 430. In at least some embodiments, the sleeve 428 has a longitudinal length 430 that is less than a length of the inner housing 402. In FIGS. 4A-4C, the sleeve 428 is shown having a longitudinal length that is equal, or substantially equal to a length of the inner housing 402. In FIGS. 4A-4C, the sleeve 428 is also shown disposed entirely within the lead lumen 412 such that the sleeve 428 does not extend outwardly from either the first end 404, or the second end 406, of the inner housing 402.

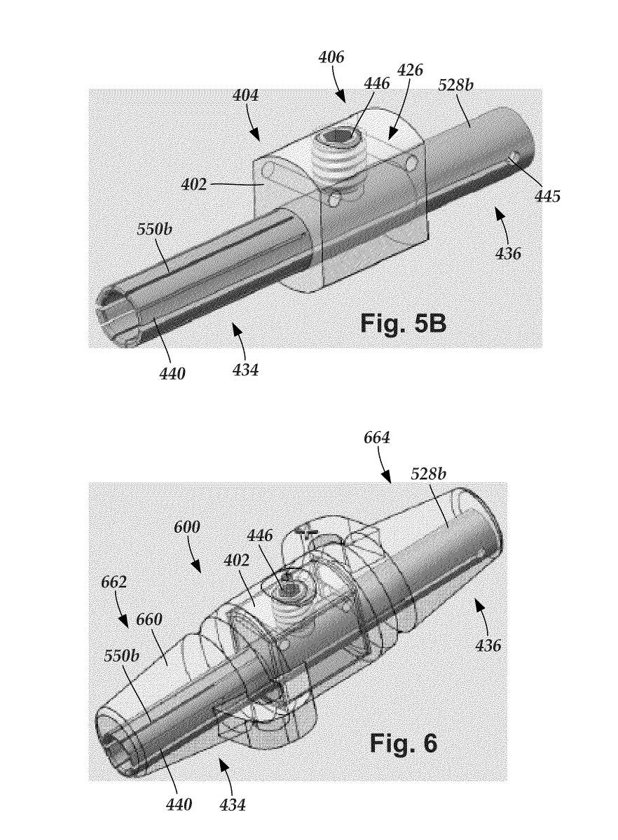

Turning to FIGS. 5A-5B, in at least some embodiments the sleeve 428 extends outwardly from the first end 404, or the second end 406, or both, of the inner housing 402. In at least some embodiments, the sleeve 428 has a longitudinal length 430 that exceeds a length of the inner housing 402. FIG. 5A illustrates, in perspective, longitudinal cross-sectional view, one embodiment of a sleeve 528a extending outwardly from each of the first end 404 and the second end 406 of the inner housing 402. FIG. 5B illustrates, in perspective view, another embodiment of a sleeve 528b extending outwardly from each of the first end 404 and the second end 406 of the inner housing 402. FIGS. 5A-5B also show the sleeves 528a, 528b each having longitudinal lengths that exceed a length of the inner housing 402.

The sleeve 528a, 528b includes the longitudinal cutout 440 extending from the edge of the first end 434 of the sleeve 528a to the terminus 445. The terminus 445 can be defined along a portion of the sleeve 528a that is disposed either within the lead lumen or external to the lead lumen. In FIG. 5A, the terminus 445 is shown defined along a portion of the sleeve 528a that is disposed within the lead lumen 412. In FIG. 5B, the terminus 445 is shown defined along a portion of the sleeve 528a that is disposed external to the lead lumen 412.

In at least some embodiments, the sleeve defines strain-relief grooves extending along one or more ends of the sleeve external to the inner housing 402. In FIG. 5A, the sleeve 528a defines strain-relief grooves 550a. Similarly, in FIG. 5B the sleeve 528b defines strain-relief grooves 550b. The strain-relief grooves can be any suitable length. The strain-relief grooves 550b of FIG. 5B are shown as being longer than the strain-relief grooves 550a. In at least some embodiments, the strain-relief grooves extend along at least 5%, 10%, 15%, 20%, 25%, 30%, 35%, 40%, 45%, 50%, or more of the longitudinal length of the sleeve.

The strain-relief grooves 550a, 550b enable some bending of the end of the sleeve relative to the inner housing, while being less flexible (i.e., more rigid) than the lead body. Thus, the bending of the sleeve along the strain-relief grooves 550a, 550b reduces potential strain exerted on the lead as the lead extends outwardly from the end of the inner housing 402 and reduces the potential for the lead to kink as it extends outwardly from the inner housing.

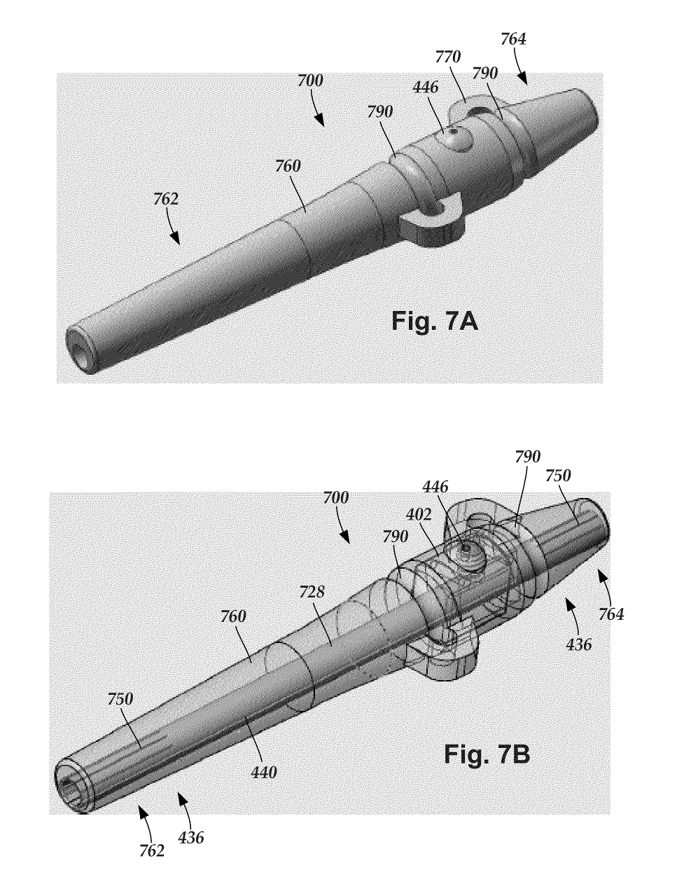

FIGS. 5A-5B both show strain-relief grooves 550a, 550b extending along the first end portion 434 of the sleeve 528a external to the inner housing 402. Alternately, the strain-relief grooves can extend from the second end portion 436 of the sleeve, or along both the first end portion 434 and the second end portion 436 of the sleeve (see e.g., FIG. 7B). At least a portion of the strain-relief grooves 550a, 550b extend externally from the inner housing 402. In at least some embodiments, the strain-relief grooves 550a, 550b are defined completely external to the inner housing 402.

The strain-relief grooves 550a, 550b can be formed in any suitable manner. In FIGS. 5A-5B, the strain-relief grooves 550a, 550b are shown as multiple grooves extending completely through the sleeve 428. In FIGS. 5A-5B, the strain-relief grooves 550a, 550b are shown circumferentially offset from one another along the lateral edge of the first end portion 434 of the sleeve and extending parallel to one another in a direction that is parallel (or substantially parallel) to the longitudinal length 430 of the sleeve 528a, 528b.

Any suitable number of strain-relief grooves may be defined around a circumference of sleeve 528a, 528b including, for example, three, four, five, six, seven, eight, nine, ten, eleven, twelve, thirteen, fourteen, fifteen, sixteen, seventeen, eighteen, nineteen, twenty, or more strain-relief grooves. The length of the strain-relief grooves 550a, 550b may be any suitable length. In at least some embodiments, the strain-relief grooves 550a, 550b have lengths that are at least 2 cm, 3 cm, 4, cm, 5 cm, 6 cm, 7 cm, 8 cm, 9 cm, 10 cm.

FIG. 6 illustrates a lead anchor 600 that includes an exterior covering 660 disposed over the inner housing 402 and the sleeve 528b. It will be understood that the sleeve 528b is shown for clarity of illustration and that the exterior covering 660 can be adapted to fit over any of the sleeves contained herein.

The exterior covering 660 provides physical protection and a watertight seal to the inner housing 402 and sleeve 528b. The exterior covering 600 has a first end 662 and an opposing second end 664. In at least some embodiments, one or more of the ends 662, 664 are elongated. In at least some embodiments, one or more of the ends 662, 664 are tapered. The exterior covering 660 may have any suitable shape including, for example, oblong, rectangular, cylindrical, elliptical, or the like, or any other regular or irregular shape, or the like. In some embodiments, the exterior covering 660 has a variable diameter that increases from one end to the middle, and then decreases from the middle to the opposite end. Alternatively, the exterior covering 660 may define a uniform diameter along all or a portion of its length.

FIG. 7A illustrates, in perspective view, another embodiment of a lead anchor 700 with an exterior covering 760. FIG. 7B illustrates, in perspective view, the lead anchor 700 with the exterior 760 shown as being transparent, for clarity of illustration. The inner housing 402 and a sleeve 728 are disposed within the exterior covering 760. The sleeve 728 shown in FIGS. 7A-7B defines the cutout 440 and strain-relief grooves 750 disposed along each of a first end portion 762 and an opposing second end portion 764 of the sleeve 728.

The exterior covering 760 may, optionally, include one or more eyelets 770 for receiving a suture, a staple, or the like, for securing the lead anchor 700 to patient tissue. The eyelets 770 may be circumferentially disposed at any suitable location around the exterior covering 760. In at least some embodiments, the eyelets 770 are 180° offset from one another along a circumference of the exterior covering 760. In at least some embodiments, the eyelets 770 are disposed on opposing end portions of the exterior covering 760. The lead anchor 700 can include any suitable number of eyelets 770 including, for example, one, two, three, four, five, six, seven, eight, or more eyelets 770. The eyelets 770 may be made from either the same material or different material from the exterior covering 660.

In at least some embodiments, the exterior covering 760 includes suture channels 790 that are disposed at least partially around a circumference of the exterior covering 760 and that are axially-aligned with the eyelets 770. The suture channels 790 facilitate suturing of the lead anchor 700 to patient tissue by enabling sutures to be disposed around the exterior covering 760 when passed through the eyelets 770 without increasing the diameter of the lead anchor 700, and while also preventing the sutures from slipping off of an end of the exterior covering 760.

In at least some embodiments, the lead anchor is designed to concurrently receive and retain multiple leads. In at least some embodiments, the lead anchor is designed to concurrently retain multiple received leads using a single fastener. FIG. 8A illustrates, in perspective view, one embodiment of an inner housing 802. FIG. 8B illustrates, in end view, one embodiment of the inner housing 802. The inner housing 802 defines multiple lead lumens, which intersect with a single fastener lumen 818 configured to receive a single fastener 846. In at least some embodiments, the fastener 846 is formed as a plug.

The inner housing 802 has a first end 804, an opposing second end 806, a top end 808, and an outer surface 810, which are similar to those described above for FIGS. 4A-5B. The inner housing 802 defines a first lead lumen 812a configured and arranged to receive a first electrical stimulation lead, and a second lead lumen 812b configured and arranged to receive a second electrical stimulation lead. The lead lumens 812a, 812b each extend across the entire inner housing 802. The first lead lumen 812a and the second lead lumen 812b may extend parallel to each other. The inner housing 802 also includes the fastener lumen 818 configured to receive the fastener 846. The fastener lumen 818 opens on the top end 808 of the inner housing 802 and intersects each of the lead lumens 812a, 812b within the inner housing 802. In FIGS. 8A-8B, the fastener lumen 818 extends between the lead lumens 812a, 812b such that the fastener lumen 818 extends transversely, or substantially transversely, to the lead lumens 812a, 812b.

The fastener 846 may have a variety of shapes for engaging concurrently with multiple leads disposed within the lead lumens 812a, 812b. In at least some embodiments, as shown in FIGS. 8A-8B, the fastener 846 may include a tapered (frustoconical) section 848 which, when the fastener 846 is moved downwardly along the fastener lumen 818 from the top end 808 of the inner housing 802, extends into the lead lumens 812a, 812b and tightens against the received lead portions for securing the lead within the lead anchor 800.

FIG. 8C illustrates, in side, perspective longitudinal cross-sectional view, one embodiment of a lead anchor 800. FIG. 8D illustrates, in top, perspective longitudinal cross-sectional view, one embodiment of the lead anchor 800. The lead anchor 800 includes an exterior covering 860 disposed over the inner housing 802 and a fastener 846. The exterior covering 860 has a first end 862 and an opposing second end 864.

In at least some embodiments, the lead anchor 800 further include a first exterior-covering lumen 866a and a second exterior-covering lumen 866b each extending across an entire length of the exterior covering 860 and opening to both the first and second ends 862, 864 of the exterior covering 860. The first exterior-covering lumen 866a may be axially-aligned with the first lead lumen 812a such that the first exterior-covering lumen 866a and the first lead lumen 812a collectively form a first continuous passageway between the first end 862 and the second end 864 of the exterior covering 860. Similarly, the second exterior-covering lumen 866b may be axially-aligned with the second lead lumen 812b such that the second exterior-covering lumen 866b and the second lead lumen 812b collectively form a second continuous passageway between the first end 862 and the second end 864 of the exterior covering 860.

Optionally, one or more sleeves can be disposed within one or more of the lead lumens, or one or more of the exterior-covering lumens, or both. FIGS. 9A-9B illustrate, transverse cross-section, one embodiment of the inner housing 802 disposed in the exterior covering 860. A first sleeve 928a is disposed in the first lead lumen 812a, and a second sleeve 928b is disposed in the second lead lumen 812b. Although not shown in FIGS. 9A-9B, one or more of the sleeves 928a, 928b may, optionally extend along at least a portion of one or more of the exterior-covering lumens 866a, 866b, respectively.

The fastener 846 is shown in FIG. 9A in a disengaged position, where one or more leads can be inserted into the lead lumens 812a, 812b. In FIG. 9B, the fastener 846 is shown in an engaged position, where the tapered section 848 of the fastener 846 is tightened against the sleeve 928a, 928b which, in turn, are tightened against the leads, when leads are received by the lead lumens 812a, 812b.

The sleeves 928a, 928b define sleeve lumens 938a, 938b, respectively, that are dimensioned to each receive an electrical stimulation lead, such as the lead 103. The sleeves 928a, 928b also define longitudinal cutouts 940a, 940b, respectively, that are reduced in width when pressed by the tapered section 848 of the fastener 846 (FIG. 9B). The sleeves 928a, 928b may, optionally, define strain-relief grooves along one end (or both ends) of the sleeves 928a, 928b.

In at least some embodiments, an electrode array of an electrical stimulation lead is advanced into a patient to a target stimulation location. The lead anchor is disposed over a portion of the lead and tightened against the lead (e.g., by screwing the fastener along the fastener aperture until the fastener presses against the sleeve with enough force to reduce the width of the longitudinal cutout of the sleeve, thereby tightening the sleeve against the received portion of the lead) such that the movement of the lead anchor causes a corresponding movement of the portion of the lead received by the lead anchor. The lead anchor is anchored to patient tissue using, for example, suture or staples (or both) passed through eyelets formed along the lead anchor. The lead anchor may be disposed over, and tightened against, a portion of the lead either before or after advancing the lead to the target stimulation location. The lead anchor may be anchored to patient tissue either before or after being disposed over, and tightened against, a portion of the lead.

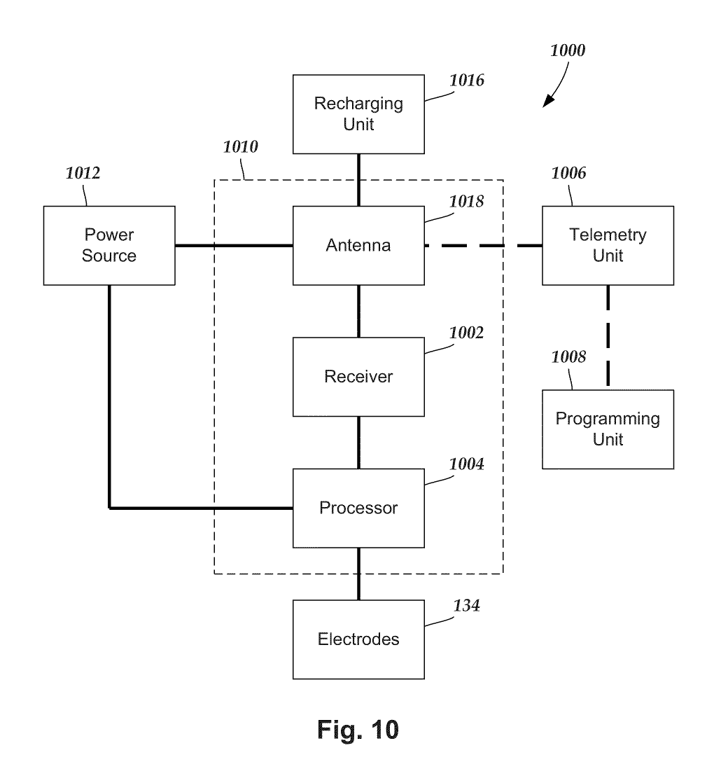

FIG. 10 is a schematic overview of one embodiment of components of an electrical stimulation system 1000 including an electronic subassembly 1010 disposed within a control module. It will be understood that the electrical stimulation system can include more, fewer, or different components and can have a variety of different configurations including those configurations disclosed in the stimulator references cited herein.

Some of the components (for example, a power source 1012, an antenna 1018, a receiver 1002, and a processor 1004) of the electrical stimulation system can be positioned on one or more circuit boards or similar carriers within a sealed inner housing of an implantable pulse generator, if desired. Any power source 1012 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 1018 or a secondary antenna. The external power source can be in a device that is mounted on the skin of the user or in a unit that is provided near the user on a permanent or periodic basis.

If the power source 1012 is a rechargeable battery, the battery may be recharged using the optional antenna 1018, if desired. Power can be provided to the battery for recharging by inductively coupling the battery through the antenna to a recharging unit 1016 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 1004 is generally included to control the timing and electrical characteristics of the electrical stimulation system. For example, the processor 1004 can, if desired, control one or more of the timing, frequency, strength, duration, and waveform of the pulses. In addition, the processor 1004 can select which electrodes can be used to provide stimulation, if desired. In some embodiments, the processor 1004 selects which electrode(s) are cathodes and which electrode(s) are anodes. In some embodiments, the processor 1004 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1008 that, for example, allows modification of pulse characteristics. In the illustrated embodiment, the processor 1004 is coupled to a receiver 1002 which, in turn, is coupled to the optional antenna 1018. This allows the processor 1004 to receive instructions from an external source to, for example, direct the pulse characteristics and the selection of electrodes, if desired.

In one embodiment, the antenna 1018 is capable of receiving signals (e.g., RF signals) from an external telemetry unit 1006 which is programmed by the programming unit 1008. The programming unit 1008 can be external to, or part of, the telemetry unit 1006. The telemetry unit 1006 can be a device that is worn on the skin of the user or can be carried by the user and can have a form similar to a pager, cellular phone, or remote control, if desired. As another alternative, the telemetry unit 1006 may not be worn or carried by the user but may only be available at a home station or at a clinician's office. The programming unit 1008 can be any unit that can provide information to the telemetry unit 1006 for transmission to the electrical stimulation system 1000. The programming unit 1008 can be part of the telemetry unit 1006 or can provide signals or information to the telemetry unit 1006 via a wireless or wired connection. One example of a suitable programming unit is a computer operated by the user or clinician to send signals to the telemetry unit 1006.

The signals sent to the processor 1004 via the antenna 1018 and the receiver 1002 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1000 to cease operation, to start operation, to start charging the battery, or to stop charging the battery. In other embodiments, the stimulation system does not include the antenna 1018 or receiver 1002 and the processor 1004 operates as programmed.

Optionally, the electrical stimulation system 1000 may include a transmitter (not shown) coupled to the processor 1004 and the antenna 1018 for transmitting signals back to the telemetry unit 1006 or another unit capable of receiving the signals. For example, the electrical stimulation system 1000 may transmit signals indicating whether the electrical stimulation system 1000 is operating properly or not or indicating when the battery needs to be charged or the level of charge remaining in the battery. The processor 1004 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A lead anchor comprising:
a inner housing having an outer surface, a top end, a first end, and a second end opposite to the first end, the inner housing defining
a lead lumen forming a continuous passageway through the inner housing, the lead lumen having a first opening defined along the first end of the inner housing and a second opening defined along the second end of the inner housing, and
a fastener lumen extending from the top end of the inner housing and forming an intersection with the lead lumen;
an exterior covering disposed over the outer surface of the inner housing; and
a lead-retention assembly configured and arranged for removably retaining an electrical stimulation lead within the lead anchor, the lead-retention assembly comprising
a sleeve formed from a rigid material and having a first end portion, an opposing second end portion, a diameter, and a longitudinal length, the sleeve disposed along surfaces of the lead lumen at the intersection between the fastener lumen and the lead lumen, the sleeve defining a sleeve lumen and at least one longitudinal cutout having a cutout width and extending along a portion of the longitudinal length of the sleeve, wherein the sleeve lumen is configured and arranged to receive the electrical stimulation lead when the electrical stimulation lead is received by the lead lumen, and
a fastener disposed in the fastener lumen, the fastener configured and arranged for retaining the received electrical stimulation lead within the lead anchor by pressing against the sleeve to reduce the diameter of the sleeve at the intersection between the fastener lumen and the lead lumen by reducing the width of the longitudinal cutout at the intersection between the fastener lumen and the lead lumen.

2. The lead anchor of claim 1, wherein the fastener is formed from a non-metallic material.

3. The lead anchor of claim 1, wherein the fastener comprises a set screw.

4. The lead anchor of claim 1, wherein the fastener comprises a plug with a tapered section.

5. The lead anchor of claim 1, wherein the at least one longitudinal cutout extends to one of the first end portion or the second end portion of the sleeve.

6. The lead anchor of claim 1, wherein the sleeve is disposed entirely within the lead lumen.

7. The lead anchor of claim 1, wherein the first end portion of the sleeve extends outwardly from the first end of the inner housing.

8. The lead anchor of claim 7, further comprising a first plurality of strain-relief grooves defined along the first end portion of the sleeve external to the inner housing.

9. The lead anchor of claim 1, wherein the second end portion of the sleeve extends outwardly from the second end of the inner housing.

10. The lead anchor of claim 9, further comprising a second plurality of strain-relief grooves defined along the second end portion of the sleeve external to the inner housing.

11. The lead anchor of claim 1, wherein the exterior covering has a first end and an opposing second end, and wherein the first end of the exterior covering has an elongated, tapered shape.

12. The lead anchor of claim 1, wherein the lead lumen is a first lead lumen, wherein the inner housing defines a second lead lumen, and wherein the first lead lumen is configured and arranged to receive a first electrical stimulation lead and the second lead lumen is configured and arranged to receive a second electrical stimulation lead.

13. The lead anchor of claim 12, wherein the first lead lumen and the second lead lumen each extend parallel to one another from the first end of the inner housing to the second end of the inner housing.

14. The lead anchor of claim 12, wherein the sleeve is a first sleeve, and wherein a second sleeve is disposed along a portion of the second lead lumen.

15. The lead anchor of claim 14, wherein the intersection between the first lead lumen and the fastener lumen is a first intersection, wherein the second lead lumen forms a second intersection with the fastener lumen, and wherein the second sleeve is disposed along a portion of the second lead lumen at the second intersection.

16. The lead anchor of claim 15, wherein the fastener is configured and arranged for simultaneously retaining the received first electrical stimulation lead within the lead anchor by pressing against the first sleeve at the first intersection and retaining the received second electrical stimulation lead within the lead anchor by pressing against the second sleeve at the second intersection.

17. An implantable stimulation device, comprising:
the lead anchor of claim 1; and
an electrical stimulation lead comprising an electrode array;
wherein the lead anchor is configured and arranged for receiving a portion of the electrical stimulation lead and removably retaining the received portion of the electrical stimulation lead.

18. The implantable stimulation device of claim 17, further comprising a control module coupleable to the electrical stimulation lead.

19. A method of implanting an implantable stimulation device, the method comprising:
providing the lead anchor of claim 1;
advancing an electrode array of a electrical stimulation lead into a patient to a target stimulation location;
extending a portion of the electrical stimulation lead along the lead lumen of the lead anchor with the electrical stimulation lead also extending through the lumen of the sleeve disposed in the lead lumen; and
tightening the fastener of the lead anchor against the sleeve to secure the electrical stimulation lead to the lead anchor, wherein tightening the fastener reduces the diameter of the sleeve by reducing the width of the slit defined along the sleeve.

20. The method of claim 19, further comprising securing the lead anchor to patient tissue in proximity to the lead anchor.
